(19) United States
(12) Patent Application Publication
(10) Pub. No.: US 2010/0175695 A1
(43) Pub. Date: Jul. 15, 2010

(54) AUXILIARY GAS MIXING IN AN ANESTHESIA SYSTEM

(75) Inventor: David T. Jamison, Quakertown, PA (US)

Correspondence Address: Mindray DS USA, Inc. c/o Stoel Rives LLP, 201 S. Main Street, Suite 1100, Salt Lake City, UT, 84111, US

(73) Assignee: MINDRAY DS USA, INC., Mahwah, NJ (US)

(21) Appl. No.: 12/352,480

(22) Filed: Jan. 12, 2009

(51) Int. Cl. A61M 16/12

(52) U.S. Cl. 128/203.14

(57) ABSTRACT

An anesthesia system includes a main output and an auxiliary output. The main output and the auxiliary output are configured to operate independently of each other. The main output may provide a first mixture of gases and an anesthetic agent to a patient. The auxiliary output may provide a second mixture of gases to the patient. A user may selectively mix oxygen and at least one other gas such that the second mixture of gases is provided to the auxiliary output at a desired flow rate and such that the second mixture of gases includes a desired oxygen percentage level. The second mixture of gases provided through the auxiliary output is prevented from flowing back into the anesthesia system to reduce or prevent contamination of the gases provided through the main output.

### TECHNICAL FIELD

The present disclosure relates to anesthesia systems.

### SUMMARY

An anesthesia system disclosed herein includes a main output and an auxiliary output. The main output and the auxiliary output are configured to operate independently of each other. The main output may provide a first mixture of gases and an anesthetic agent to a patient. The auxiliary output may provide a second mixture of gases to the patient. In certain embodiments, a user may selectively mix oxygen and at least one other gas, such that the second mixture of gases is provided to the auxiliary output at a desired flow rate and includes a desired oxygen percentage level. In addition, or in other embodiments, the second mixture of gases provided through the auxiliary output is prevented from flowing back into the anesthesia system to reduce or prevent contamination of the gases provided through the main output.

### BRIEF DESCRIPTION OF THE DRAWINGS

FIG. 1 is a block diagram of an anesthesia system according to one embodiment;

FIG. 2 is a block diagram illustrating further details of the anesthesia system shown in FIG. 1 according to one embodiment;

FIG. 3 is a block diagram of an auxiliary gas control subsystem according to one embodiment;

FIG. 4 is a block diagram of an auxiliary gas control subsystem according to another embodiment;

FIG. 5 is a block diagram illustrating an auxiliary gas control subsystem that includes an electronic gas blender according to one embodiment;

FIG. 6 is a schematic diagram graphically illustrating a physical layout of an anesthesia system according to one embodiment;

FIG. 7 is a schematic diagram graphically representing a GUI for an anesthesia system according to one embodiment; and

### DETAILED DESCRIPTION

During surgical procedures, there is typically a need to anesthetize a patient in order to reduce or eliminate any pain associated with the procedure. A breathing system for administering an inhaled anesthetic agent may include, for example, an anesthesia machine in which the anesthetic agent is provided in a flow of carrier gas. The carrier gas is usually a mixture of oxygen and nitrous oxide and/or air. The carrier gas and anesthetic agent from the anesthesia machine are provided to a main breathing machine or anesthetic circuit for delivery to a patient during the patient's respiration.

An oxygen source may be used for both the anesthesia machine and an auxiliary output provided directly to the patient. The auxiliary output may be provided to the patient through a nasal cannula and may be used, for example, before and/or after surgery to help stabilize the patient, or at other times when the patient is able to breath on her or his own. Many procedures use the auxiliary gas outlet as the only gas provided to the patient.

Providing approximately 100% oxygen through the auxiliary output, however, may be hazardous. For example, multiple studies have shown that fires in operating rooms may be caused on occasion by the buildup of oxygen in the area around a nasal cannula, in the patient's airway, under drapes, and/or under masks. This oxygen-enriched atmosphere creates an environment in which objects burn more readily and robustly than in room air (e.g., approximately 21% oxygen). Further, ignition sources (e.g., electrosurgical units, lasers, electrocautery pencil tips, bronchoscope lights, and fiberoptic light sources) may often be used in an operating room environment. Electrosurgical units or lasers used to cut and coagulate tissue present particular risks during airway surgeries. Such ignition sources may easily start fires in oxygen enriched environments. Thus, there is a need to reduce or eliminate the risks associated with providing approximately 100% oxygen through the auxiliary output of an anesthesia system.

As discussed in detail below, an anesthesia system according to one embodiment disclosed herein includes an auxiliary gas control subsystem that allows a user to mix oxygen with air inside the anesthesia system to selectively adjust the percentage of oxygen delivered to a patient and reduce the likelihood of a fire caused by ignition of a combustible material in an oxygen enriched environment. While the auxiliary gas control subsystem accesses the same oxygen and air sources used by the anesthesia machine and provided to the patient through the main breathing system, the auxiliary gas control subsystem operates independently of the anesthesia machine such that either may be used even if the other is not operational. Further, the auxiliary gas control subsystem is configured to reduce the likelihood or prevent the gas sources (e.g., oxygen and air) within the anesthesia machine from contaminating each other during the mixing of the auxiliary gas such that the overall system may continue to provide precise gas blends.

The embodiments of the disclosure will be best understood by reference to the drawings, wherein like elements are designated by like numerals throughout. In the following description, numerous specific details are provided for a thorough understanding of the embodiments described herein. However, those of skill in the art will recognize that one or more of the specific details may be omitted, or other methods, components, or materials may be used.

Furthermore, the described features, operations, or characteristics may be combined in any suitable manner in one or more embodiments. It will also be readily understood that the order of the steps or actions of the methods described in connection with the embodiments disclosed may be changed as would be apparent to those skilled in the art. Thus, any order in the drawings or Detailed Description is for illustrative purposes only and is not meant to imply a required order, unless specified to require an order.

Embodiments may include various steps, which may be embodied in machine-executable instructions to be executed by a general-purpose or special-purpose computer (or other electronic device). Alternatively, the steps may be performed by hardware components that include specific logic for performing the steps or by a combination of hardware, software, and/or firmware.

Embodiments may also be provided as a computer program product including a machine-readable medium having stored thereon instructions that may be used to program a computer (or other electronic device) to perform processes described herein. The machine-readable medium may include, but is not limited to, hard drives, floppy diskettes, optical disks, CD-ROMs, DVD-ROMs, ROMs, RAMs, EPROMS, EEPROMs, magnetic or optical cards, solid-state memory devices, or other types of media/machine-readable medium suitable for storing electronic instructions.

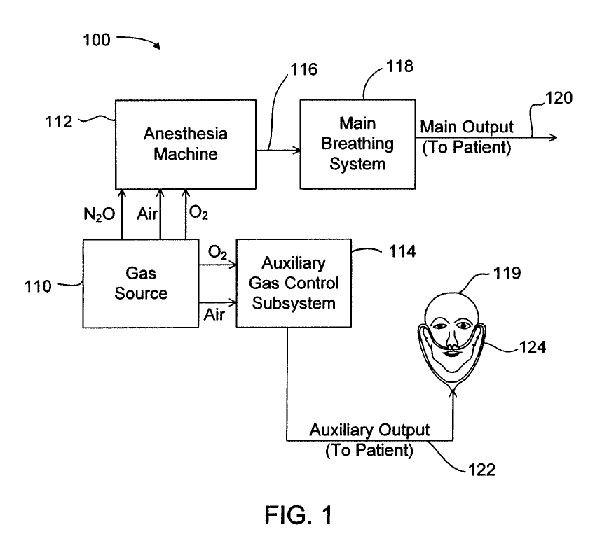

FIG. 1 is a block diagram of an anesthesia system 100 according to one embodiment. The system 100 includes a gas source 110 configured to provide breathable gases to an anesthesia machine 112 and an auxiliary gas control subsystem 114. The breathable gases may include, for example, oxygen (O₂), nitrous oxide (N₂O), and medical air. The gas source 110 may include a plurality of compressed gas cylinders, a plurality of gas supply lines (e.g., connections to piped hospital oxygen, medical air, and/or nitrous oxide), or both.

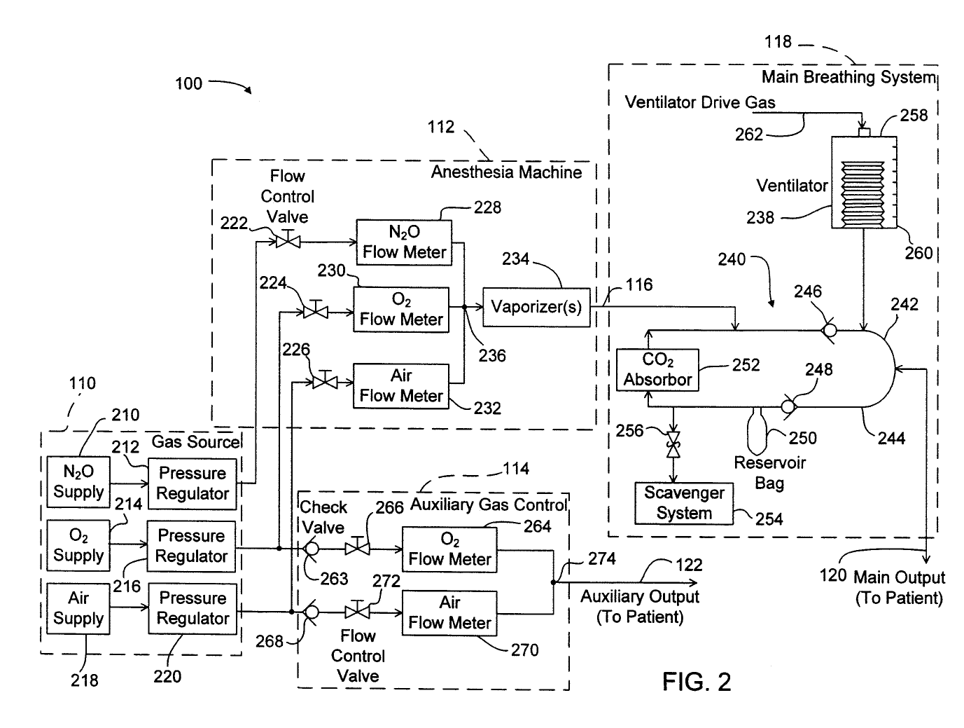

Although not shown in FIG. 1, the anesthesia machine 112 may include pressure regulators, gas flow control valves, and flow meters to selectively mix the breathable gases from the gas source 110 in desired ratios. The anesthesia machine 112 may also include one or more vaporizers (see FIG. 2) to accurately add an anesthetic agent to the mix of breathable gases. The anesthesia machine 112 discharges the mixed gases having the anesthetic agent therein through a common output 116 to a main breathing system 118. Although not shown in FIG. 1, the main breathing system 118 may include a ventilator, a carbon dioxide (CO₂) absorber, a reservoir bag, a scavenger system to remove excess gases, a warmed breathing system, a bacteria filter, and/or a humidifier. As is understood by those skilled in the art, the main breathing system 118 according to certain embodiments may be operated in a variety of ventilator modes including, for example, pressure controlled ventilation (PCV), pressure support ventilation (PS), synchronized intermittent mandatory ventilation (SIMV), and volume controlled ventilation (CMV). When desired, the main breathing system 118 provides the breathable gases and anesthetic agent to a patient 119 through a main output 120, typically through an endotracheal tube or laryngeal mask airway.

The auxiliary gas control subsystem 114 operates independently of the anesthesia machine 112 and the main breathing system 118 to selectively provide a separate mixture of gases (e.g., O₂ and air) to the patient 119 through an auxiliary output 122. The auxiliary output 122 may interface with the patient 119 through, for example, a nasal cannula 124. A person of ordinary skill in the art will recognize, however, that the auxiliary output 122 may interface with the patient 119 through other types of devices (e.g., a mask). By integrating the functions of the auxiliary gas control subsystem 114 with the gas source 110, anesthesia machine 112, and main breathing system 118, a user may easily and selectively provide between approximately 21% (e.g., approximately 100% air) to approximately 100% O₂ from the auxiliary gas output 122. Thus, the embodiments disclosed herein increase safety and reduce the chances of operating room fires.

As discussed in detail below, the auxiliary gas control subsystem 114 is configured to reduce or prevent the combined auxiliary gases from re-entering the gas source 110. For example, if the pressure of the air is higher than that of the O₂, a portion of the air may flow back from the auxiliary gas control subsystem 114 to the O₂ cylinder or supply line in the gas source 110. Such contamination may cause problems with accurate gas delivery in the main anesthesia machine 112. Thus, the auxiliary gas control subsystem 114 according to certain embodiments prevents or reduces the likelihood of any gases returning to the gas source 110.

In one embodiment, the user may blend the auxiliary gas provided through the auxiliary output 122 to, for example, approximately 100% O₂, 100% air, 50% O₂ and 50% air, or any other ratio of O₂ and air, by visually noting the flow rate for each gas and manually adjusting a flow control valve (not shown) for each gas. As discussed below, in certain such embodiments, the auxiliary gas control subsystem 114 may also include an oxygen sensor to provide an indication to the user of the O₂ percentage level being provided to the patient 119 through the auxiliary output 122. Other sensors may also be used such as sensors for measuring the patient's blood oxygen level or exhaled CO₂ level.

In addition, or in other embodiments, the auxiliary gas control subsystem 114 may provide automatic flow rate adjustment of either or both gases to achieve a desired O₂ percentage level through the auxiliary output 122. For example, the user may simply enter a desired O₂ percentage level and an overall flow rate, and the auxiliary gas control subsystem 114 may make the necessary adjustments to achieve the selected parameters. In certain such embodiments, an oxygen sensor (not shown) may provide feedback in a closed loop control system to automatically maintain the desired O₂ percentage level. Those skilled in the art will recognize from the disclosure herein that other types of feedback may also be used to dynamically adjust the gas flow rates. For example, the adjustments may be based on measurements of the patient's blood oxygen and/or exhaled CO₂ level. Similar types of feedback and control may also be used for the main output 120 provided by the anesthesia machine 112 and the main breathing system 118. Further, in certain embodiments, the anesthesia machine 112 and/or the main breathing system 118 may share one or more sensors with the auxiliary gas control subsystem 114. For example, an input to an O₂ sensor may be selectively or automatically switched between the main output 120 and the auxiliary output 122.

Although FIG. 1 shows the gas source 110 of the anesthesia system 100 providing only O₂ and air to the auxiliary gas control subsystem 114, the disclosure herein is not so limited. For example, the auxiliary gas control subsystem 114 may be used to blend O₂ with other types of gases such as nitrogen, nitrous oxide, nitric oxide, helium, and/or xenon. In addition, or in other embodiments, the auxiliary gas control subsystem 114 may mix more than one gas with O₂. Further, the auxiliary gas control subsystem 114 may also provide an anesthetic agent and/or medications through the auxiliary output 122 to the patient 119. For example, the auxiliary gas control subsystem 114 may combine the auxiliary gases with an inhalant such as an anti-inflammatory steroid and/or a bronchodilator for patients with asthma. A person skilled in the art will recognize from the disclosure herein that the auxiliary gas control subsystem 114 may provide a variety of other medications and gases not specifically mentioned by way of example herein.

FIG. 2 is a block diagram illustrating further details of the anesthesia system 100 shown in FIG. 1 according to one embodiment. As discussed above, the system includes a main output 120 and an auxiliary output 122 for selectively providing breathable gases, anesthetic agents, and/or medications to a user 119 (see FIG. 1). In the embodiment shown in FIG. 2, the gas source 110 includes an N₂O supply 210 connected to a pressure regulator 212, an O₂ supply 214 connected to a pressure regulator 216, and an air supply 218 connected to a pressure regulator 220. Each gas supply 210, 214, 218 may include a compressed gas cylinder and/or a gas supply line (e.g., a connection to a piped gas line). In one embodiment, each supply 210, 214, 218 provides its respective gas at a pressure between approximately 280 kPa and approximately 600 kPa. A person skilled in the art will recognize, however, that many other different pressures may also be used. The pressure regulators 212, 216, 220, when used, reduce the pressure of their respective gases to safe pressures or pressures that are usable by the anesthesia machine and the auxiliary gas control subsystem 114.

The anesthesia machine according to the embodiment shown in FIG. 2 includes flow control valves 222, 224, 226, flow meters 228, 230, 232, and one or more vaporizers 234. In one embodiment, the user manually adjusts the flow control valves 222, 224, 226 to adjust the flow of N₂O, O₂, and air, respectively, based on visual indications of flow rates (e.g., in liters/minute) displayed by the respective flow meters 228, 230, 232. In one embodiment, the flow meters 228, 230, 232 include ball flow meters. In other embodiments, the flow meters 228, 230, 232 may include electronic flow meters. The gases (e.g., N₂O, O₂, and air) from the gas source 110 are combined at a location 236 upstream from the one or more vaporizers 234. The selected vaporizer 234 combines an anesthetic agent with the mixed gases and discharge the gases and anesthetic agent through the common output 116 to the main breathing system 118.

The main breathing system 118 according to the embodiment shown in FIG. 2 includes a ventilator 238 connected to a respiration loop 240 that includes an inhalation limb 242 and an exhalation limb 244 connected to the main output 120. The gases and anesthetic agent from the common output 116 of the anesthesia machine 112 enter the respiration loop 240, which includes an inhalation check valve 246 connected to the inhalation limb 242, an expiration check valve connected to the exhalation limb 244, a reservoir bag 250 downstream from the exhalation check valve 248, and a CO₂ absorber 252 between the reservoir bag 250 and the inhalation check valve 246. The main breathing system 118 also includes a scavenger system 254 connected to the respiration loop 240 through a pressure relief or pop-off valve 256. A person skilled in the art will recognize that the elements displayed in the main breathing circuit are provide by way of example only and may be arranged in a different order.

In one embodiment, the ventilator 238 includes an expandable, pleated bellows 258 contained in a housing 260 that is sealed except for an opening for a ventilator drive gas 262. The ventilator drive gas 262 enters and exits the housing 260 to drive the bellows 258 up and down so as to force the gases and anesthetic agent around the loop 240 and out the main output 120. When the patient exhales, exhaled gases return to the loop 240 through the main output 120 and pass through the exhalation check valve 248 to the reservoir bag 250. The reservoir bag 250 is expandable and contractible in response to the divergence of gas flow therein. The reservoir bag 250 may therefore be used as a visual indicator of the patient's respiration. When the pressure reaches a predetermined level in the loop 240, the pop-off valve 256 opens to allow the scavenger system 254 to capture excess gases. The CO₂ absorber may include, for example, soda lime or other suitable CO₂ absorbent materials. The fresh gases from the common output 116 of the anesthesia machine 112 flow through the inhalation check valve 246 and are inhaled by the patient through the main output 120.

The auxiliary gas control subsystem 114 according to the embodiment shown in FIG. 2 includes a check valve 263 that receives the O₂ gas from the gas source 110 and provides the O₂ gas to an O₂ flow meter 264 through a flow control valve 266. A person skilled in the art will recognize that the check valve 263, the flow control valve 266, and the O₂ flow meter 264 may be arranged in a different order. The auxiliary gas control subsystem 114 also includes a check valve 268 that receives the air from the gas source 110 and provides the air to an air flow meter 270 through a flow control valve 272. A person skilled in the art will recognize that the check valve 268, the flow control valve 272, and the air flow meter 270 may be arranged in a different order.

In one embodiment, the O₂ flow meter 264 and the air flow meter 270 each comprise a ball flow meter. In other embodiments, the flow meters 264, 270 may include electronic flow meters. As the user adjusts the flow control valves 266, 272, the user can determine the flow rate of the O₂ gas by observing the response of the O₂ flow meter 264 and the flow rate of the air by observing the response of the air flow meter 270. The O₂ gas and the air combine at a point 274 downstream from the O₂ flow meter 264 and the air flow meter 270. By observing both meters 264, 270, the user can determine the overall flow rate and the O₂ percentage level of the mixed gas provided through the auxiliary output 122.

The check valves 263, 268 are configured to prevent gases from flowing from the auxiliary gas control module 114 back to the gas source 110 or into the anesthesia machine 112. Thus, for example, air from the air supply 218 is prevented from flowing from the point 274 where the gases are combined back to the O₂ supply 214 or to the flow control valve 224 in the anesthesia machine 112. Similarly, gas from the O₂ supply 214 is prevented from flowing from the point 274 where the gases are combined back to the air supply 218 or to the flow control valve 226 in the anesthesia machine 112. This allows the anesthesia machine 112 to independently continue providing precise gas blends without concern that the O₂ gas and/or the air provided to the anesthesia machine 112 has been cross contaminated.

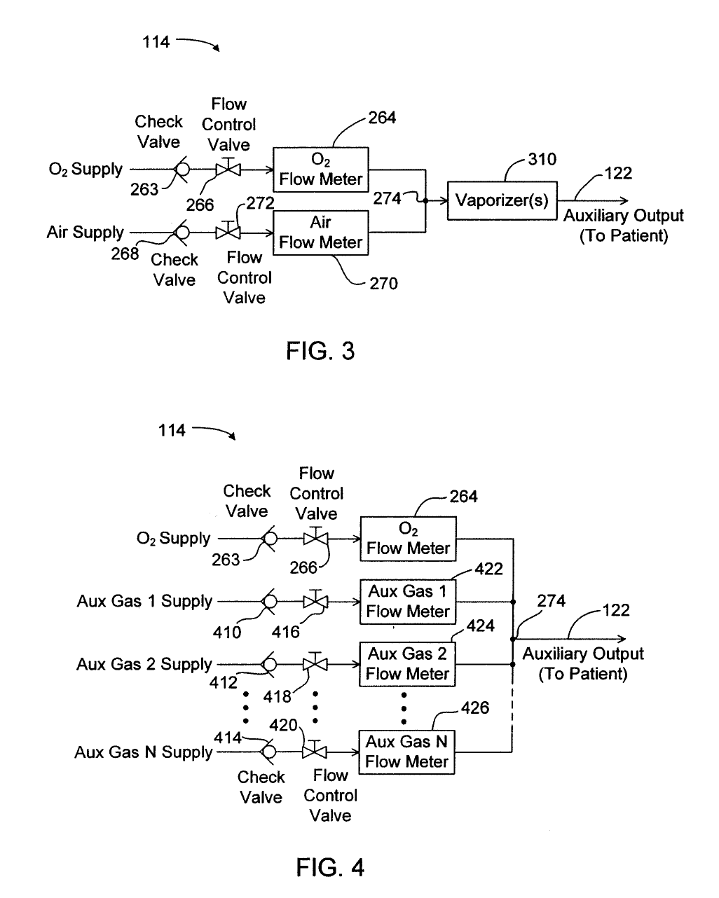

FIG. 3 is a block diagram of an auxiliary gas control subsystem 114 according to another embodiment. The auxiliary gas control subsystem 114 shown in FIG. 3 includes the check valves 263, 268, the flow control valves 266, 272, and the flow meters 264, 270 discussed above in relation to FIG. 2. However, the auxiliary gas control subsystem 114 shown in FIG. 3 also includes one or more vaporizers 310 for adding an anesthetic agent and/or medication to the auxiliary gases before they are provided to the patient via the auxiliary output 122, as discussed above.

FIG. 4 is a block diagram of an auxiliary gas control subsystem 114 according to another embodiment. The auxiliary gas control subsystem 114 shown in FIG. 4 includes the check valve 263, the flow control valve 266, and the O₂ flow meter 264 shown and discussed above in relation to FIG. 2. However, the auxiliary gas control subsystem 114 shown in FIG. 4 also includes a plurality of additional check valves 410, 412, 414, flow control valves 416, 418, 420, and auxiliary gas flow meters 422, 424, 426. Thus, the auxiliary gas control subsystem 114 shown in FIG. 4 may combine two or more gases with the O₂ gas for output to the patient via the auxiliary output 122. For example, the user may configure the auxiliary gas control subsystem 114 shown in FIG. 4 to selectively mix air, nitrogen, nitrous oxide, nitric oxide, helium, xenon, and/or other gases or medications with the O₂ gas. By visually observing the flow meters 264, 422, 424, 426, the user may adjust the appropriate flow control valves 266, 416, 418, 420 to achieve a desired ratio of gases and overall flow rate. In addition, or in other embodiments, the auxiliary gas control subsystem 114 shown in FIG. 4 may include one or more vaporizers 310, as shown in FIG. 3, to combine an anesthetic agent and/or medication with the auxiliary gases.

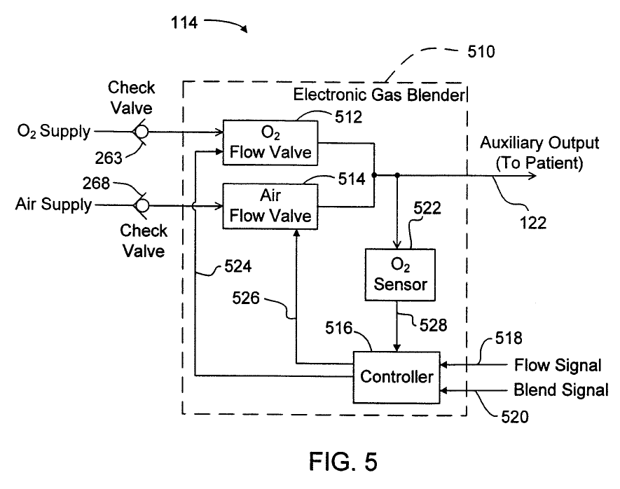

In certain embodiments, the auxiliary flow control valves 266, 272 and the auxiliary flow meters 264, 270 shown in FIG. 2 are replaced by a mechanical or electronic gas blender to increase the precision of the mixed gas ratios. For example, FIG. 5 is a block diagram illustrating an auxiliary gas control subsystem 114 that includes an electronic gas blender 510 according to one embodiment. The auxiliary gas control subsystem 114 shown in FIG. 5 also includes the check valves 263, 268 and the auxiliary output 122 discussed above.

The electronic gas blender 510 includes an O₂ electronic flow valve 512 connected to the O₂ supply through the check valve 263, an air electronic flow valve 514 connected to the air supply through the check valve 268, and a controller 516 for independently controlling the flow of gases through the O₂ flow valve 512 and the air flow valve 514 based on a flow signal 518 and a blend signal 520.

The flow signal 518 is selected by the user to specify the overall flow rate of the combined gases provided to the auxiliary output 122. The blend signal 520 is selected by the user to specify an O₂ percentage level of the mixed gases provided to the auxiliary output 122. Based on these two signals, the controller 516 determines respective flow rates for the O₂ flow valve 512 and the air flow valve 514. The controller 516 provides a first control signal 524 to the O₂ flow valve 512 to adjust a passage way (not shown) therein according to the calculated O₂ flow rate. Similarly, the controller 516 provides a second control signal 526 to the air flow valve 514 to adjust a passage way (not shown) therein according to the calculated air flow rate. Thus, the overall flow rate and the O₂ percentage provided to the auxiliary output 122 can be accurately and automatically controlled.

In certain embodiments, the electronic gas blender 510 also includes an O₂ sensor 522 that samples a portion of the gas combined at the output of the O₂ flow valve 512 and the air flow valve 514. The O₂ sensor 522 provides a signal 528 to the controller 516 that represents the O₂ percentage level currently being provided in the blended auxiliary gases through the auxiliary output 122. In certain embodiments, the controller 516 displays the O₂ percentage level so that the user can make any necessary adjustments. In addition, or in other embodiments, the controller 516 uses the sensed O₂ percentage level as feedback to adjust the control signals 524, 526 provided to the flow valves 512, 514 so as to obtain and/or maintain the desired O₂ percentage level provided to the auxiliary output 122.

Although not shown, the controller 516 may include, for example, a microprocessor, a memory device comprising software code for causing the microprocessor to perform the functions described herein, an analog to digital converter for interfacing the output of the O₂ sensor with the microprocessor, and driver circuitry for controlling the O₂ flow valve 512 and the air flow valve 514.

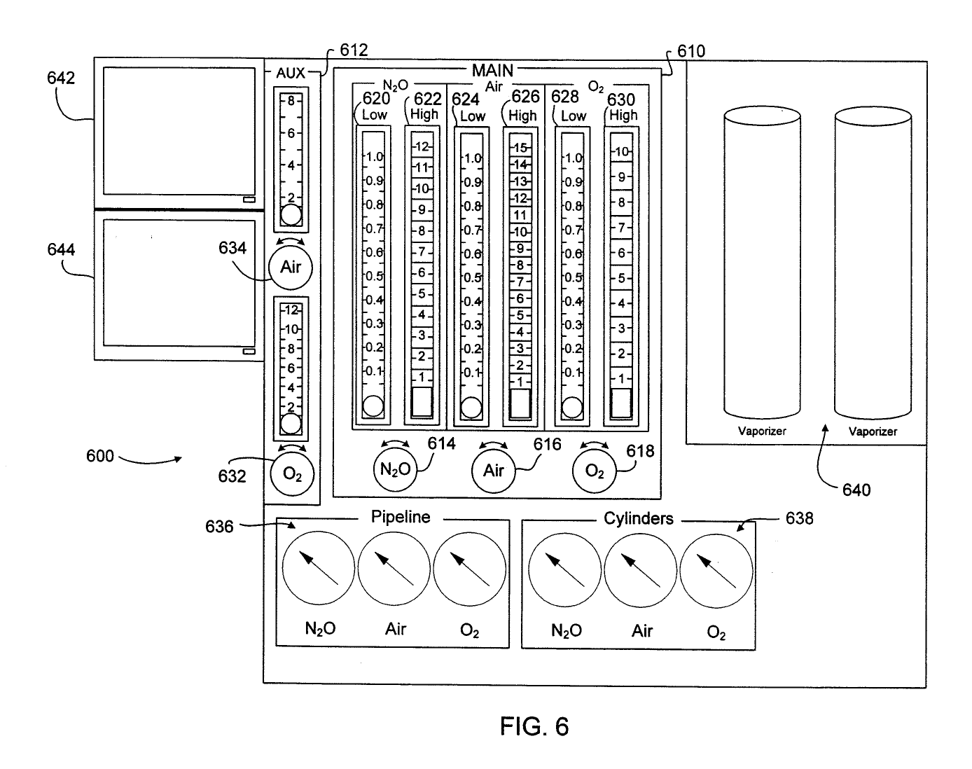

FIG. 6 is a schematic diagram graphically illustrating a physical layout 600 of an anesthesia system, such as the anesthesia system 100 shown in FIG. 2, according to one embodiment. As shown in the example embodiment of FIG. 6, the layout 600 includes a main section 610 for controlling the main output 120 and an auxiliary section 612 for controlling the auxiliary output 122 shown in FIG. 2. The main section 610 includes a user control 614 for adjusting the flow control valve 222 to control the flow rate of N₂O flowing into the anesthesia machine 112, a user control 616 for adjusting the flow control valve 226 to control the flow rate of air flowing into the anesthesia machine, and a user control 618 for adjusting the flow control valve 224 to control the flow rate of O₂ flowing into the anesthesia machine. While the user controls 614, 616, 618 are shown as knobs or dials, a person of skill in the art will recognize from the disclosure herein that other configurations may be used for the user controls 614, 616, 618 such as sliders or levers.

As shown in FIG. 2, each flow control valve 222, 224, 226 is connected to a corresponding flow meter 228, 230, 232. In the example embodiment shown in FIG. 6, however, a high range and a low range flow meter is used for each gas in the anesthesia machine 112. The N₂O flows from the flow control valve 222 to a low range flow meter 620 and a high range flow meter 622, the air flows from the control valve 226 to a low range flow meter 624 and a high range flow meter 626, and the O₂ flows from the flow control valve 224 to a low range flow meter 628 and a high range flow meter 630.

In this example embodiment, the high range N₂O flow meter 622 measures gas flow rates in a range between approximately 1 liter/minute and approximately 12 liters/minute, the high range air flow meter 626 measures gas flow rates in a range between approximately 1 liter/minute and approximately 15 liters/minute, and the high range O₂ flow meter 630 measures gas flow rates in a range between approximately 1 liter/minute and approximately 10 liters/minute. The low range flow meters 620, 624, 628 in this example each measure gas flow rates in a range between approximately 0.05 liter/minute and approximately 1 liter/minute. A person of skill in the art will recognize from the disclosure herein that any other gas flow rate may also be measured. Thus, the user is allowed to accurately monitor the flow rate of each gas flowing through the anesthesia machine 112 as the user makes desired adjustments to the flow control valves 222, 224, 226 using the appropriate user controls 614, 616, 618.

The auxiliary section 612 includes a user control 632 for adjusting the flow control valve 266 to control the flow rate of O₂ flowing in the auxiliary gas control subsystem 114, and a user control 634 for adjusting the flow control valve 272 to control the flow rate of air flowing in the auxiliary gas control subsystem 114. While the user controls 632, 634 are shown as knobs or dials, a person of skill in the art will recognize from the disclosure herein that other configurations may be used for the user controls 632, 634 such as sliders or levers.

FIG. 6 also illustrates the positions of the O₂ flow meter 264 and the air flow meter 270 of the auxiliary gas control subsystem 114 shown in FIG. 2. In this example embodiment, the O₂ flow meter 264 measures gas flow rates in a range between approximately 1 liter/minute and approximately 12 liters/minute, and the air flow meter 270 measures gas flow rates in a range between approximately 1 liter/minute and approximately 8 liters/minute. A person of skill in the art will recognize from the disclosure herein that any other gas flow rate may also be measured for the gases in the auxiliary gas control subsystem 114. Thus, the user is allowed to monitor the flow rate of each gas flowing through the auxiliary gas control subsystem 114 as the user makes desired adjustments to the flow control valves 266, 272 using the appropriate user controls 632, 634.

As shown in FIG. 6, the physical layout 600 of the anesthesia system may also include a plurality of pressure gauges 636 for gases (N₂O, air, O₂) provided through centralized pipelines and/or a plurality of pressure gauges 638 for gases (N₂O, air, O₂) of pressurized cylinders, both of which may be part of the gas source 110 discussed above in relation to FIG. 2. The physical layout 600 of FIG. 6 also provides, by way of example only, locations of one or more replaceable vaporizer canisters 640 corresponding to the vaporizers 234 shown in FIG. 2. FIG. 6 also illustrates example locations of a first monitor 642 and a second monitor 644. The first monitor 642 may display, for example, monitored patient data such as, without limitation, heart rate, blood pressure, electrocardiogram data, blood oxygen level, and so forth. The second monitor 644 may display, for example, ventilator information such as graphics corresponding to airway pressure and flow, tidal volume, minute volume, peak airway pressure, positive end expiratory pressure, mean pressure, plateau pressure, breath rate, FiO₂, and so forth.

While the physical layout 600 shown in FIG. 6 corresponds to the physical positions of the flow control valves 222, 224, 226, 266, 272, the flow meters 228, 230, 232, 264, 270, and the vaporizers 234 discussed in relation to FIG. 2, a person of skill in the art will recognize from the disclosure herein that a corresponding layout may be provided on a graphical user interface (GUI) displayed on a local or remote display screen. For example, the user controls 614, 616, 618, 632, 634 and flow meters 620, 622, 624, 626, 628, 630, 264, 270, and pressure gauges 636, 638 may each be represented graphically on a display screen. Further, in certain embodiments, the vaporizer canisters 640 may graphically represent the amount of anesthetic agent remaining in the vaporizers 234 shown in FIG. 2.

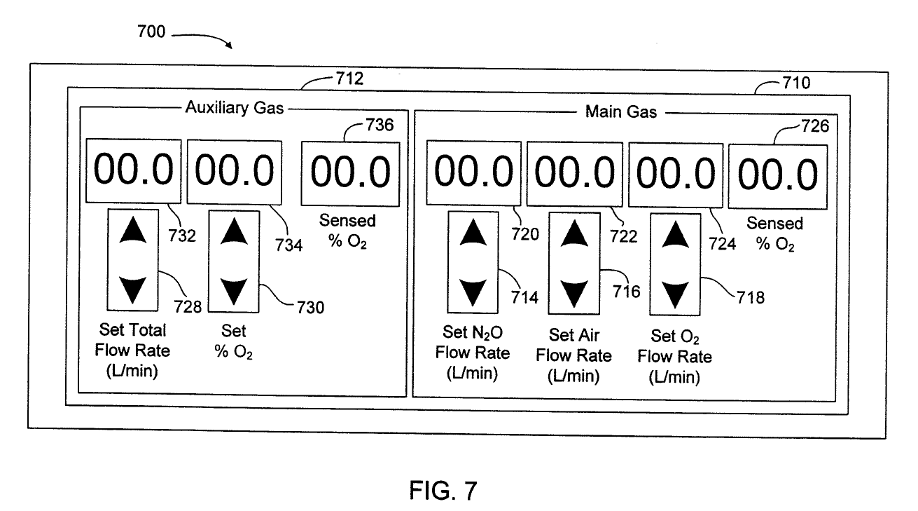

FIG. 7 is a schematic diagram graphically representing a GUI 700 for an anesthesia system according to another embodiment. The GUI 700 may be used, for example, with electronic gas blenders, such as the electronic gas blender 510 shown in FIG. 5, to control and graphically represent the flow of gases in the system. The GUI 700 displays a main gas section 710 and an auxiliary gas section 712.

The main gas section 710 graphically represents a user control 714 to set the N₂O flow rate, a user control 716 to set the air flow rate, and a user control 718 to set the O₂ flow rate. The main gas section 710 also graphically represents a flow indicator 720 for displaying the flow rate of N₂O, a flow indicator 722 for displaying the flow rate of air, and a flow indicator 724 for displaying the flow rate of O₂. The gas flow rates may be measured, for example, using electronic flow meters (not shown). The main gas section 710 may also graphically represent an indicator 726 for displaying the percentage of O₂ sensed in the main output 120 of the anesthesia system.

The auxiliary gas section 712 graphically represents a user control 728 to set the total flow rate for the O₂ and air provided by the auxiliary output 122. The auxiliary gas section 712 also graphically represents a user control 730 to set the percentage of O₂ provided in the auxiliary output 122. The auxiliary gas section 712 also graphically represents a flow indicator 732 for displaying the total flow rate selected by the user, an indicator 734 for displaying the percentage of O₂ selected by the user, an indicator 736 for displaying the percentage of O₂ sensed in the auxiliary output 122 of the anesthesia system, and an indicator 738 for displaying the total flow rate sensed in the auxiliary output 122 of the anesthesia system. Thus, as discussed above, a user may simply select the desired flow rate and percentage of O₂, and the auxiliary gas control subsystem 114 automatically makes the necessary adjustments without further input by the user to provide the desired gas mixture through the auxiliary output 122.

In other embodiments, the anesthesia system may indicate to the user the appropriate O₂ flow setting and/or the appropriate air flow setting to achieve a desired O₂ percentage level at a desired overall flow rate. For example, a table (not shown) indicates appropriate flow settings to achieve a desired O₂ percentage level and overall flow rate according to one embodiment. The table may be graphically displayed on a screen or printed on a card attached to the anesthesia machine. For example, the table may indicate respective O₂ and air flow settings (e.g., in liters/minute) to achieve a 30% O₂ concentration in the auxiliary output 122 for a variety of total flow rates (2, 4, 6, 9, 13, or 17 liters/minute). To achieve a total flow rate of 6 liters/minute at 30% O₂, for example, the user may manually adjust the flow control valve 266 to provide approximately 0.7 liters/minute of O₂ (as indicated by the O₂ flow meter 264) and the flow control valve 272 to provide approximately 5.3 liters/minute of air (as indicated by the air flow meter 270). Of course, the disclosure herein is not limited to 30% O₂. Indeed, settings may be provided for any combination of O₂ percentage level and total flow rates. Further, the user may be able to select between multiple displayable tables (or between multiple cards with printed tables) to find settings for a desired O₂ percentage level and overall flow rate. In other embodiments, a user is allowed to select the desired O₂ level and desired overall flow rate (e.g., using dials or controls such as the controls 728, 730 shown in FIG. 7), and the anesthesia machine displays (e.g., using dials or digital displays) the appropriate O₂ and air flow rate settings that the user may use to adjust the flow control valves 266, 272.

Although not shown, in certain embodiments the anesthesia system 100 disclosed herein includes a wired or wireless communication system to provide remote monitoring and/or control of the gases provided through the main output 120 and/or the auxiliary output 122. Thus, the GUI 700 shown in FIG. 7 (or another GUI, e.g., a GUI having the example layout 600 shown in FIG. 6) may be used either locally in the presence of the anesthesia machine 100 or from a remote location. In addition, or in other embodiments, the GUI 700 may be provided to a portable device such as, but not limited to, a laptop computer, a personal digital assistant, a cell phone, or another portable device configured to communicate directly with the communication system of the anesthesia system 100 or through a network.

It will be understood to those having skill in the art that many changes may be made to the details of the above-described embodiments without departing from the underlying principles of the invention. The scope of the present invention should, therefore, be determined only by the following claims.

1. A system comprising:
a main output configured to provide a first mixture of gases and an anesthetic agent to a patient;
an auxiliary output configured to provide a second mixture of gases to the patient, wherein the main output and the auxiliary output are configured to operate independently of each other;
an anesthesia machine configured to selectively mix oxygen from an oxygen source and one or more other gases so as to provide a first flow rate and a first oxygen percentage for the first mixture of gases, and to combine the anesthetic agent with the first mixture of gases; and
an auxiliary gas control subsystem configured to selectively mix oxygen from the oxygen source and one or more other gases so as to provide a second flow rate and a second oxygen percentage for the second mixture of gases provided through the auxiliary output.

2. The system of claim 1, wherein the auxiliary gas control subsystem comprises:
a first check valve configured to allow oxygen from the oxygen source to flow into the auxiliary gas control subsystem, and to substantially prevent a reverse flow of gases from the auxiliary gas control subsystem to the oxygen gas source; and
a second check valve configured to allow a second gas from a second gas source from flowing into the auxiliary gas control subsystem, and to substantially prevent a reverse flow of gases from the auxiliary gas control subsystem to the second gas source.

3. The system of claim 2, wherein the auxiliary gas control subsystem further comprises:
a first flow control valve configured to selectively control a flow rate of the oxygen through the auxiliary gas control subsystem;
a second flow control valve configured to selectively control a flow rate of the second gas through the auxiliary gas control subsystem,
wherein second flow rate and the second oxygen percentage for the second mixture of gases are determined at least in part by the flow rate of the oxygen selected by the first flow control valve and the flow rate of the second gas selected by the second flow control valve.

4. The system of claim 3, wherein the auxiliary gas control subsystem further comprises:
a first flow meter configured to indicate the flow rate of the oxygen selected by the first flow control valve; and
a second flow meter configured to indicate the flow rate of the second gas selected by the second flow control valve.

5. The system of claim 2, wherein the auxiliary gas control subsystem further comprises an electronic gas blender comprising:
a controller configured to:
receive a user-selectable flow signal corresponding to the second flow rate;
receive a user-selectable blend signal corresponding to the second oxygen percentage;
provide a first control signal corresponding to a flow rate of the oxygen based on the flow signal and the blend signal; and
provide a second control signal corresponding to a flow rate of the second gas based on the flow signal and the blend signal;
a first electronic control valve configured to control the flow rate of the oxygen through the auxiliary control subsystem based on the first control signal from the controller; and
a second electronic control valve configured to control the flow rate of the second gas through the auxiliary control subsystem based on the second control signal from the controller.

6. The system of claim 5, further comprising an oxygen sensor configured to measure the oxygen percentage provided by the electronic gas blender to the auxiliary output.

7. The system of claim 6, wherein the oxygen sensor provides feedback to the controller, and wherein the controller is configured to adjust at least one of the first control signal and the second control signal based on the feedback from the oxygen sensor.

8. The system of claim 5, further comprising a graphical user interface displayable on a remote terminal, the graphical user interface configured to allow a user to selectively set the flow signal and the blend signal.

9. The system of claim 1, wherein the anesthesia machine comprises:
a plurality of flow control valves configured to selectively control the flow rates of the oxygen and the one or more other gases, wherein the first flow rate and the first oxygen percentage of the first mixture of gases provided to the main output are determined at least in part by the plurality of flow control valves in the anesthesia machine;
a plurality of flow meters configured to indicate the flow rates of the oxygen and the one or more other gases within the anesthesia system; and
one or more vaporizers configured to combine the anesthetic agent with the first mixture of gases.

10. The system of claim 1, further comprising a main breathing system configured to receive the first mixture of gases and the anesthetic agent from the anesthesia machine and to provide the anesthetic agent to the main output.

11. The system of claim 10, wherein the main breathing system comprises:
a ventilator configured to assist breathing, at least in part, by the patient through the main output;
a carbon dioxide absorber in a respiration loop configured to remove carbon dioxide exhaled by the patient from the main breathing system; and
a scavenger system configured to remove excess gases from the main breathing system.

12. The system of claim 1, wherein the one or more other gases selectively mixed with the oxygen by the auxiliary gas control subsystem are selected from the group comprising air, nitrogen, nitrous oxide, nitric oxide, helium, and xenon.

13. The system of claim 1, wherein the auxiliary gas control subsystem is further configured to deliver an anesthetic agent to the patient through the auxiliary output.

14. The system of claim 1, wherein the auxiliary gas control subsystem is further configured to deliver one or more medications to the patient through the auxiliary output.

15. A method for selectively providing mixed gases to a patient through a first output and/or a second output of an anesthesia system, the method comprising:
forming a first gas mixture by selectively mixing a first gas from a first source with a second gas from a second source;
combining the first gas mixture with an anesthetic agent;
providing the first gas mixture and the anesthetic agent through the first output; and
independent of mixing or providing the first gas mixture:
forming a second gas mixture by selectively mixing the first gas from the first source with at least one of the second gas from the second gas source and a third gas from a third gas source; and
providing the second gas mixture through the second output.

16. The method of claim 15, wherein forming the second gas mixture comprises selecting a flow rate of the first gas from the first source and a flow rate of the second gas from the second source to provide a desired overall flow rate of the second gas mixture and a desired oxygen percentage of the second gas mixture.

17. The method of claim 15, further comprising:
allowing the first gas to flow from the first source toward the second output; and
substantially preventing gases from flowing from the second output toward the first source or the first output.

18. The method of claim 17, further comprising:
allowing the second gas to flow from the second source toward the second output; and
substantially preventing gases from flowing from the second output toward the second source or the first output.

19. The method of claim 15, further comprising:
providing indication to a user of a selected flow rate of the first gas forming part of the second gas mixture; and
providing indication to the user of a selected flow rate of the second gas forming part of the second gas mixture.

20. The method of claim 15, further comprising:
receiving a user-selectable flow signal corresponding to a desired overall flow rate of the second gas mixture;
receiving a user-selectable blend signal corresponding to a desired oxygen percentage of the second gas mixture; and
automatically adjusting, based on the flow signal and the blend signal and without further user intervention, a flow rate of the first gas from the first source and a flow rate of the second gas from the second source to provide the desired overall flow rate of the second gas mixture and the desired oxygen percentage of the second gas mixture.

21. The method of claim 15, further comprising sensing an oxygen level of the second gas mixture provided through the second output.

22. The method of claim 21, further comprising automatically adjusting, based on the sensed oxygen level and without further user intervention, a flow rate of the first gas from the first source and a flow rate of the second gas from the second source to provide a desired overall flow rate of the second gas mixture and a desired oxygen percentage of the second gas mixture.

23. A system for selectively providing mixed gases to a patient through a first output and/or a second output of an anesthesia system, the system comprising:
means for forming a first gas mixture by selectively mixing a first gas from a first source with a second gas from a second source;
means for combining the first gas mixture with an anesthetic agent;
means for providing the first gas mixture and the anesthetic agent through the first output; and
means, independent of the means for forming or means for providing the first gas mixture, for:
forming a second gas mixture by selectively mixing the first gas from the first source with at least one of the second gas from the second gas source and a third gas from a third gas source; and
providing the second gas mixture through the second output.

24. The system of claim 23, wherein the means for forming the second gas mixture comprises means for selecting a flow rate of the first gas from the first source and a flow rate of the second gas from the second source to provide a desired overall flow rate of the second gas mixture and a desired oxygen percentage of the second gas mixture.

25. The system of claim 23, further comprising means for allowing the first gas to flow from the first source toward the second output, and for substantially preventing gases from flowing from the second output toward the first source or the first output.

26. The system of claim 25, further comprising means for allowing the second gas to flow from the second source toward the second output, and for substantially preventing gases from flowing from the second output toward the second source or the first output.

27. The system of claim 23, further comprising:
means for providing indication to a user of a selected flow rate of the first gas forming part of the second gas mixture; and
means for providing indication to the user of a selected flow rate of the second gas forming part of the second gas mixture.

28. The system of claim 23, further comprising:
means for receiving a user-selectable flow signal corresponding to a desired overall flow rate of the second gas mixture;
means for receiving a user-selectable blend signal corresponding to a desired oxygen percentage of the second gas mixture; and
means for automatically adjusting, based on the flow signal and the blend signal and without further user intervention, a flow rate of the first gas from the first source and a flow rate of the second gas from the second source to provide the desired overall flow rate of the second gas mixture and the desired oxygen percentage of the second gas mixture.

29. The system of claim 23, further comprising means for sensing an oxygen level of the second gas mixture provided through the second output.

30. The system of claim 29, further comprising means for automatically adjusting, based on the sensed oxygen level and without further user intervention, a flow rate of the first gas from the first source and a flow rate of the second gas from the second source to provide a desired overall flow rate of the second gas mixture and a desired oxygen percentage of the second gas mixture.
